(19) United States
(12) Patent Application Publication
(10) Pub. No.: US 2024/0079098 A1
(43) Pub. Date: Mar. 7, 2024

(54) DEVICE FOR PREDICTING DRUG-TARGET INTERACTION BY USING SELF-ATTENTION-BASED DEEP NEURAL NETWORK MODEL, AND METHOD THEREFOR

(71) Applicant: GWANGJU INSTITUTE OF SCIENCE AND TECHNOLOGY, Gwangju (KR)

(72) Inventors: Ho Jung Nam, Gwangju (KR); In Goo Lee, Gwangju (KR)

(21) Appl. No.: 18/274,433

(22) PCT Filed: Nov. 29, 2021
(86) PCT No.: PCT/KR2021/017765

§ 371 Date: Jul. 26, 2023

(30) Foreign Application Priority Data
Feb. 1, 2021 (KR) 10-2021-0014357

(51) Int. Cl. G16C 20/70; G16C 20/50; G16C 20/90

(52) U.S. Cl. CPC G16C 20/70; G16C 20/50; G16C 20/90

(57) ABSTRACT

The present invention relates to drug-target protein interaction prediction using deep learning, and a device and a method for predicting a drug-target interaction (DTI), according to the present invention, train a transformer network by using the interaction between a drug and a protein, and the binding region of the drug and the protein, and predict DTI and the binding region by using the transformer network using an attention score, and thus DTI prediction performance can be increased.

### TECHNICAL FIELD

The present invention relates to drug-target interaction prediction, and more particularly, to drug-target interaction prediction using artificial intelligence.

### BACKGROUND ART

In a biotechnology research method, a method of conducting an experiment based on living organisms is called ‘in-vivo’, and a method through a glass test tube is called ‘in-vitro’.

The case of testing drug response in cells cultured in test tubes or laboratory animals faces ethical problems as well as time and cost problems. Therefore, recently, an in-silico method of predicting drug interaction based on computer simulations, rather than actual living organisms or cells, has been attempted.

Identifying drug-target interaction (DTI) is a very important step in discovering new drugs. Since the number of drugs is infinite, it is impossible to try all possible drugs against target proteins.

Therefore, a method of predicting drugs applicable to a target protein in a drug database by using an in-silico method has become a method capable of increasing the efficiency of drug discovery. In particular, attempts to predict DTI by using deep learning have been made as drug databases have recently accumulated and computing power has increased.

However, since a convolutional neural network (CNN), a recursive neural network (RNN), and a transformer-based artificial intelligence model do not explicitly train a binding region (BR) of a drug, there is a limitation on accuracy of prediction.

The inventors of the present invention have made research efforts to overcome the limitation of these prior art drug-target interaction prediction methods. The present invention has been made in much effort to complete a device and method for DTI prediction, in which a self-attention technique is combined with a CNN to predict a binding region and DTI of a drug and a protein target together, thereby increasing the accuracy of DTI and binding region prediction.

### DISCLOSURE OF INVENTION

### Technical Problem

An object of the present invention is to provide a device and method for drug-target interaction (DTI) prediction, in which a binding region where a drug binds to a target protein is predicted and reflected to DTI, thereby increasing the accuracy of DTI and binding region prediction.

On the other hand, other unspecified objects of the present invention will be additionally considered within the scope that can be easily inferred from the following detailed description and effects thereof.

### Technical Solution

A method for predicting drug-target interaction by using a self-attention-based deep neural network, according to the present invention, includes:

(a) training a transformer network by a drug fingerprint and a protein sequence database; (b) transforming the drug fingerprint into a drug token by passing the drug fingerprint through a dense layer; (c) transforming a protein sequence into a protein grid encoding by performing a convolution operation on the protein sequence, dividing the protein sequence into predetermined unit grids, and performing max pooling thereon; (d) concatenating the drug token to the protein grid encoding; (e) inputting the drug token and the protein grid encoding, which are concatenated to each other, to the transformer network; and (f) predicting an interaction between a drug and a target protein by an output of the transformer network.

The drug fingerprint is a Morgan fingerprint hashed by a Morgan algorithm.

The drug fingerprint and the protein sequence database in the step (a) includes a three-dimensional structure and binding information of the drug and the protein.

In the step (a), the transformer network is trained by transforming a binding site of the binding information into a binding region including up to a sequence adjacent to the binding site.

The step (c) includes performing a convolutional operation on the protein sequence by using a Convolution Neural Network (CNN)

The drug token and the unit grid have a same length.

The step (e) includes transforming the drug token and the protein grid encoding, which are concatenated to each other, into Q (query), K (key), and V (value) vectors and inputting the Q (query), K (key), and V (value) vectors to the transformer network.

The transformer network includes two or more transformer networks.

The step (f) includes predicting a relationship between the drug and the protein by using an attention score between the drug and the protein grid encoding.

### Advantageous Effects

According to the present invention, it is possible to increase the accuracy of drug-target interaction (DTI) prediction by not simply predicting DTI, but also predicting a binding region where a drug binds to a target protein and reflecting the result to the DTI.

On the other hand, even when the effects are not explicitly mentioned herein, it is noted that the effects described in the following specification expected by the technical features of the present invention and their provisional effects are treated as described in the specification of the present invention.

### BRIEF DESCRIPTION OF DRAWINGS

FIG. 1 is a schematic configuration diagram of a device for predicting drug-target interaction according to a preferred embodiment of the present invention.

FIG. 2 is an example of a binding region according to a preferred embodiment of the present invention.

FIG. 3 is an example of transformation of drug data and protein sequence according to a preferred embodiment of the present invention.

FIG. 4 is an example of an operation of a transformer network according to a preferred embodiment of the present invention.

FIG. 5 is an example of an output of a transformer network according to a preferred embodiment of the present invention.

FIG. 6 is a graph showing the performance of a device for predicting drug-target interaction according to a preferred embodiment of the present invention.

FIG. 7 is a flowchart of a method for predicting drug-target interaction according to another preferred embodiment of the present invention.

It is noted that the accompanying drawings are illustrated as references for understanding the technical idea of the present invention, and the scope of the present invention is not limited thereby.

### BEST MODE FOR CARRYING OUT THE INVENTION

Hereinafter, the configuration of the present invention guided by various embodiments of the present invention and the effects resulting from the configuration will be described with reference to the drawings. In describing the present invention, when relevant known functions obvious to those of ordinary skill in the art are determined to unnecessarily obscure the gist of the present invention, a detailed description thereof will be omitted.

While the terms such as “first” ‘and “second” may be used to describe various elements’, such elements should not be limited by these terms. These terms are only used to distinguish one element from another. For example, while not departing from the scope of the present invention, a ‘first element’ may be referred to as a ‘second element’, and similarly, a ‘second element’ may be referred to as a ‘first element’. In addition, the singular forms as used herein are intended to include the plural forms as well unless the context clearly indicates otherwise. Terms used in the embodiments of the present invention may be interpreted as meanings commonly known to those of ordinary skill in the art unless otherwise defined.

Hereinafter, the configuration of the present invention guided by various embodiments of the present invention and the effects resulting from the configuration will be described with reference to the drawings.

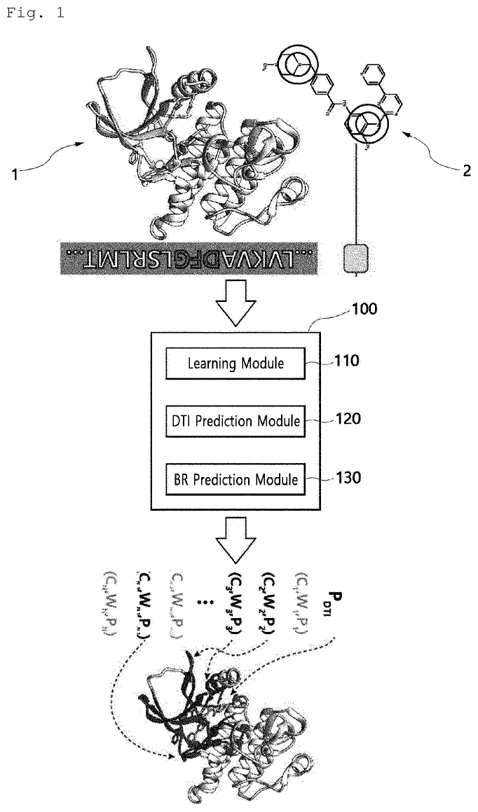

FIG. 1 is a schematic configuration diagram of a device for predicting drug-target interaction (DTI) according to a preferred embodiment of the present invention.

A device 100 for predicting DTI according to the present invention includes a learning module 110, a DTI prediction module 120, and a binding region (BR) prediction module 130.

According to the present invention, DTI and BR may be predicted after passing through an artificial neural network by using protein sequence data 1 and drug fingerprint data 2 as inputs.

To this end, the artificial neural network uses a transformer network. The transformer network may find out the relationship between a drug and a protein or between proteins by using a self-attention method. Based on this, DTI and BR may be predicted. Therefore, a deep learning model of the present invention may be referred to as Highlight on Target Sequence (HoTS).

First, the learning module 110 trains the transformer network. The transformer network is trained by a DTI database and a 3D binding structure database of drugs and proteins. For training, a step of transforming a binding site into a BR is required.

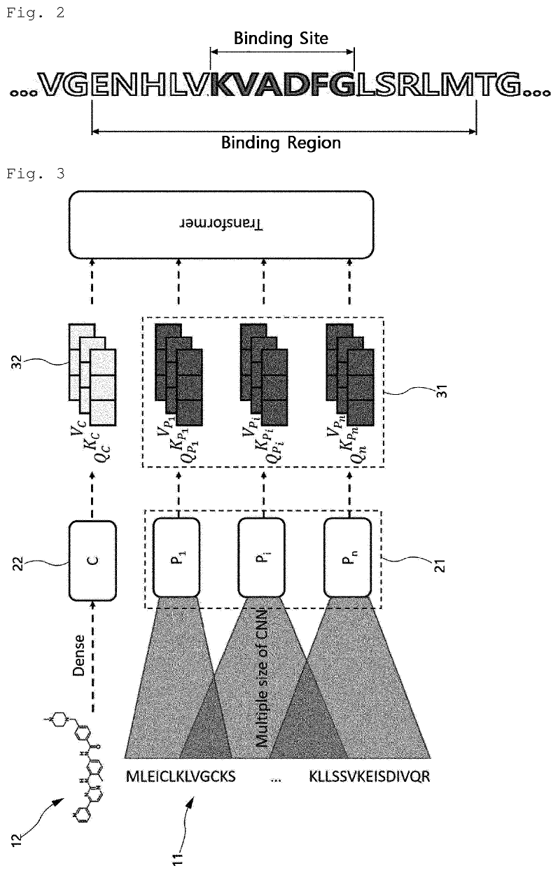

FIG. 2 is an example of a BR transformation according to a preferred embodiment of the present invention.

Since the size of the binding site of protein is very small, it is difficult to recognize the binding site in an artificial neural network. Therefore, a certain region on the protein sequence about 2-3 times the size of the binding site is set as the BR and then used for training.

A method for training a transformer network for a predictive model is as follows.

First, a fingerprint of a drug is transformed into a vector for transformer network input. The fingerprint of the drug may be represented as a Morgan fingerprint through a Morgan algorithm. The Morgan fingerprint may be represented by 2048 bits of radius of 2. The Morgan fingerprint is transformed into a drug token vector of a certain length by passing through a dense layer, that is, a fully connected layer.

The protein sequence is convoluted by using a Convolution Neural Network (CNN). The convolution operation result has the same length as the original protein sequence. The operation result is divided into grids of certain units, and the maximum values are extracted from the grids (Max Pooling). The extracted maximum values are transformed into protein grid encoding by passing through the dense layer. It is more effective at predicting BR and model interdependency.

The drug token vector and the protein grid encoding are concatenated to each other and input to the transformer network, whereby the transformer network is trained. The drug token refers to DTI, and the protein grid encoding predicts the ligand and its selectivity, that is, the BR.

The BR prediction module 130 predicts the BR by predicting the relationship between the drug token and a specific part of the protein.

As in the previous example, the drug fingerprint is transformed into the drug token and the protein is transformed into the protein grid encoding. Then, the drug token and the protein grid encoding are input to the transformer network.

FIG. 3 is an example of transformation of drug data and protein sequence according to a preferred embodiment of the present invention.

A Morgan fingerprint 12, which is a drug fingerprint, passes through a dense layer and is transformed into a drug token 22.

A protein sequence 11 passes through a convolution operation and a dense layer and is transformed into a protein grid encoding 21 through max pooling.

Each of the drug token 22 and the protein grid encoding 21 is transformed into Q (query), K (key), and V (value) vectors 31 and 32 by a weight matrix and is input to a transformer network.

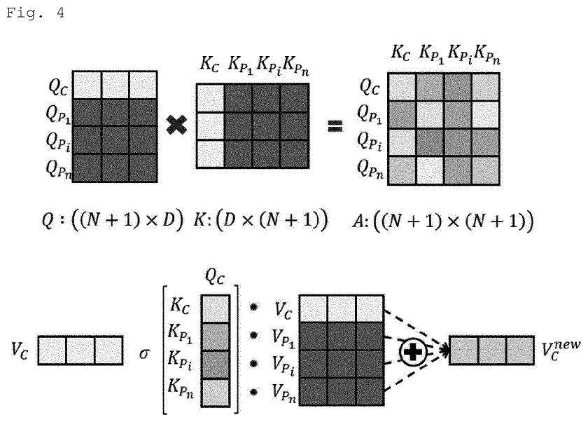

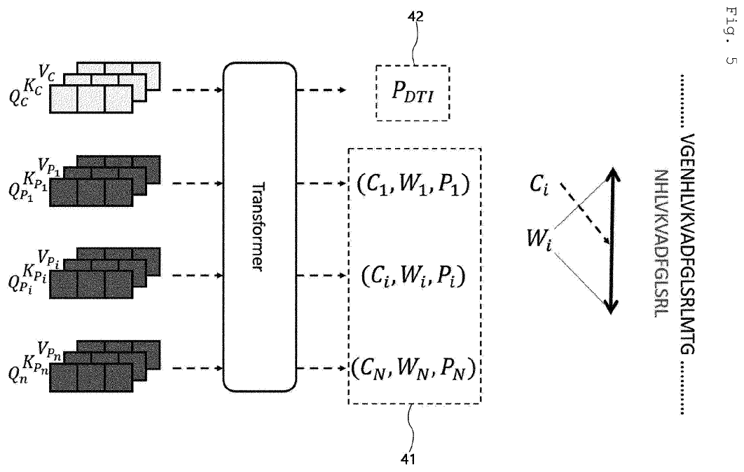

FIG. 4 is an example of an operation of a transformer network according to a preferred embodiment of the present invention.

A resulting matrix A of (N+1) rows X (N+1) columns is calculated by multiplying a matrix including (N+1) Q vectors with a D length by a matrix including (N+1) K vectors, and a new V vector is calculated by multiplexing the matrix A by (N+1) V vectors with the D length.

The calculated V vector may be used for DTI calculation, and the calculated grid vector can be used for ligand selectivity, that is, BR prediction.

FIG. 5 is an example of an output of a transformer network according to a preferred embodiment of the present invention.

The BR prediction module 130 predicts the BR by using the output of the transformer network. An output 41 of the protein grid encoding is (C, W, P).

In the (C, W, P) pair, C refers to the center of the predicted BR, W refers to the width of the BR, and P refers to the binding confidence score. Therefore, as the P value increases, the probability that the corresponding part is the BR increases.

(C, W, P) passes through the dense layer from the protein grid encoding and is activated by using an activation function. As the activation function, a sigmoid function or the like may be used. Therefore, (C, W, P) has a value between [0, 1].

A C(C_{g}) value is changed to a center value (Center_{g}) of the predicted protein BR through the following equation.

Center_{g}=S_{g}+size_{grid}*C_{g}

Here, S_{g} is a starting index of the protein grid and size_{grid} is a size of the grid.

Similarly, a W(W_{g}) value is changed to the width of the predicted protein BR through the following equation.

Width_{ig}=rᵢ*e^{Wg}

Here, rᵢ is a predetermined size and e is a natural constant. As an example, if 10, the predicted width ranges between [10, 27].

The DTI prediction module 120 predicts whether the drug token interacts with the protein.

In order to predict the interaction between the drug and the protein, the drug token and the protein grid encoding are input to the transformer network in the same manner as described above.

In the transformer network, the drug token is the sum of the product of the protein grid encodings and the attention score of the protein grid encoding for the drug encoding. After passing through the dense layer and the activation function, the drug token has a value between [0, 1]. Therefore, a final output 42 of the drug token in FIG. 5 means the probability of the DTI, and the DTI may be predicted by this probability.

As described above, the device for predicting DTI according to the present invention trains the BR of the drug and the protein as well as the interaction between the drug and the protein, and predicts the DTI and the BR by using this learning, thereby improving DTI prediction performance.

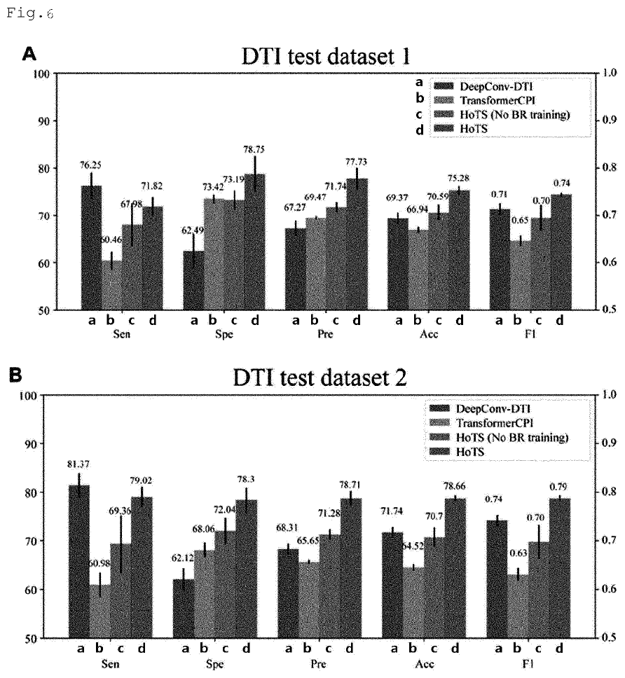

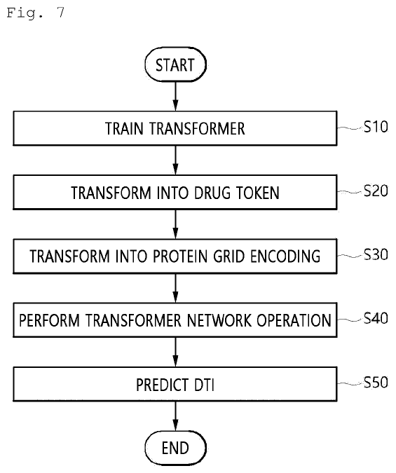

FIG. 6 is a graph showing the performance of a device (HoTS) for predicting DTI according to the present invention

It can be seen that the performance of the device (HoTS) for predicting DTI according to the present invention is higher than the performance of devices using other methods. In particular, even in the prediction device according to the present invention, the performance of the device that trains the BR is better than the performance of the device that does not train the BR (No BR Training). Accordingly, it can be seen that training and predicting the BR together has a better effect on the performance of DTI.

FIG. 7 is a summary of a flowchart of a method for predicting DTI according to another preferred embodiment of the present invention.

First, the transformer network to be used for predicting DTI according to the present invention should be trained (S10).

The training of the transformer network uses the drug fingerprint database and the protein sequence database. By training the BR as well as the DTI between the drug and the protein, the BR may be predicted and the DTI performance may be improved.

After training the transformer network, the interaction between the drug and the protein may be predicted.

Morgan fingerprint may be used as the drug fingerprint and is transformed into the drug token vector of a certain length by passing through the dense layer, that is, the fully connected layer (S20).

The protein sequence is convoluted by using a CNN. The convolution operation result has the same length as the original protein sequence. The operation result is divided into grids of certain units, and the maximum values are extracted from the grids (Max Pooling). The extracted maximum values are transformed into protein grid encoding by passing through the dense layer (S30).

The transformed drug token and the protein grid encoding are input to the pre-trained transformer network, and transformer network operation is performed (S40). At this time, the transformer network may include two or more transformer networks.

Finally, the DTI and the BR are predicted by the output of the transformer network (S50).

The final output of the drug token means the probability of the DTI, and the DTI may be predicted by this probability.

The final output of protein grid encoding includes (C, W, P), C refers to the center of the predicted BR, W refers to the width of the BR, and P refers to the binding confidence score. It is possible to predict the BR in the protein sequence.

As described above, the device and method for predicting DTI according to the present invention train the BR of the drug and the protein as well as the interaction between the drug and the protein, and predict the DTI and the BR by using the transformer network that uses the self-attention method, thereby improving DTI prediction performance.

The protection scope of the present invention is not limited to the explicit description and expression of the embodiments. In addition, it is noted again that the protection scope of the present invention cannot be limited due to obvious changes or substitutions in the technical field to which the present invention belongs.

### INDUSTRIAL APPLICABILITY

The method for predicting DTI by using the self-attention-based deep neural network according to the present invention can be used in various fields such as new drug development and biotechnology research.

1. A method for predicting a binding region or drug-target interaction by using a self-attention-based deep neural network, the method being performed by a control unit including one or more processors and a memory, the method comprising:
(a) training a transformer network by a drug fingerprint and a protein sequence database;
(b) transforming the drug fingerprint into a drug token by passing the drug fingerprint through a dense layer;
(c) transforming a protein sequence into a protein grid encoding by performing a convolution operation on the protein sequence, dividing the protein sequence into predetermined unit grids, and then performing max pooling thereon;
(d) concatenating the drug token to the protein grid encoding;
(e) inputting the drug token and the protein grid encoding, which are concatenated to each other, to the transformer network; and
(f) predicting an interaction between a drug and a target protein or a binding region where the drug binds to the target protein by an output of the transformer network.

2. The method of claim 1, wherein the drug fingerprint is a Morgan fingerprint hashed by a Morgan algorithm.

3. The method of claim 1, wherein the drug fingerprint and the protein sequence database in the step (a) comprise a three-dimensional structure and binding information of the drug and the protein.

4. The method of claim 3, wherein, in the step (a), the transformer network is trained by transforming a binding site of the binding information into a binding region including up to a sequence adjacent to the binding site.

5. The method of claim 1, wherein the step (c) comprises performing a convolutional operation on the protein sequence by using a Convolution Neural Network (CNN).

6. The method of claim 1, wherein the drug token and the unit grid have a same length.

7. The method of claim 1, wherein the step (e) comprises transforming the drug token and the protein grid encoding, which are concatenated to each other, into Q (query), K (key), and V (value) vectors and inputting the Q (query), K (key), and V (value) vectors to the transformer network.

8. The method of claim 1, wherein the transformer network comprises two or more transformer networks.

9. The method of claim 1, wherein the step (f) comprises predicting a relationship between the drug and the protein by using an attention score between the drug and the protein grid encoding.
